(19) United States
(12) Patent Application Publication
(10) Pub. No.: US 2021/0127978 A1
(43) Pub. Date: May 6, 2021

(54) OPTICAL TOMOGRAPHY DEVICE

(71) Applicants: HUAN-CHENG CHANG, Taoyuan City (TW); National Central University, Taoyuan City (TW)

(72) Inventors: Min-Chun PAN, Taoyuan City (TW); Pet-Ting LIU, New Taipei City (TW); Ya-Fen HSU, Taoyuan City (TW)

(21) Appl. No.: 16/693,350

(22) Filed: Nov. 24, 2019

(30) Foreign Application Priority Data
Nov. 4, 2019 (TW) 108139977

(51) Int. Cl. A61B 5/00

(52) U.S. Cl. CPC A61B 5/0073; A61B 2562/0242; A61B 5/0091

(57) ABSTRACT

An optical tomography device includes a carrier base, at least one slide rail, at least one sliding assembly, and at least one optical channel member. The carrier base has a through hole. The slide rail is located on the carrier base and extends toward the through hole. The sliding assembly includes a sliding block, a guiding rod, and an elastic component. The sliding block is slidably connected to the slide rail and has a first restriction portion. The guiding rod is slidably connected to the sliding block and has a second restriction portion. The elastic component is configured to be deformed by the first restriction portion and the second restriction portion. The optical channel member is coupled with the guiding rod.

### CROSS-REFERENCE TO RELATED APPLICATION

This application claims priority to Taiwan Application Serial Number 108139977, filed Nov. 4, 2019, which is herein incorporated by reference in its entirety.

### BACKGROUND

### Field of Invention

The present disclosure relates to an optical tomography device.

### Description of Related Art

Due to changes in pollution, diet and lifestyle, cancer has gradually become one of the top causes of human death in modern society. Therefore, the prevention and detection of cancer has gradually become the research target for experts and scholars.

Breast cancer is one of the most common types of cancers, and it is the number one cancer incidence rate among women. The earlier findings of breast cancer have a considerable impact on the treatment effect. The 5-year survival rate of earlier breast cancer is over 90%, and the survival rate of the first stage breast cancer is as high as 95%. Therefore, the detection of breast cancer is quite important for the treatment of breast cancer.

Breast cancer has a variety of detection methods, and optical tomography is one of the most common methods. Because an optical tomography machine has a fixed size, each patient may have different sizes of breasts, it may cause discomfort to the patient in breast cancer detection. In addition, the obtained imaging by such optical tomography will inevitably lead to errors in the results, resulting in a decrease in the accuracy of breast cancer detection results.

Therefore, there is a need of providing an optical tomography device that can solve the above problems.

### SUMMARY

One aspect of the present disclosure is to provide an optical tomography device includes a carrier base, at least one slide rail, at least one sliding assembly, and at least one optical channel member. The carrier base has a through hole. The slide rail is located on the carrier base and extends toward the through hole. The sliding assembly includes a sliding block, a guiding rod, and an elastic component. The sliding block is slidably connected to the slide rail and has a first restriction portion. The guiding rod is slidably connected to the sliding block and has a second restriction portion. The elastic component is configured to be deformed by the first restriction portion and the second restriction portion. The optical channel member is coupled with the guiding rod.

In one or more embodiments, the sliding block has a third restriction portion, the guiding rod is configured to penetrate the first and third restriction portions, and slidably connected with the first and third restriction portions.

In one or more embodiments, the sliding block has an inner cavity to accommodate the elastic component.

In one or more embodiments, the sliding block has a guiding slot communicated with the inner cavity and a protruding member slidably connected within the guiding slot.

In one or more embodiments, the guiding slot is configured to extend toward the through hole.

In one or more embodiments, the at least one sliding assembly further includes a fastener located at an end of the guiding rod that is immediately-adjacent to the through hole, the fastener has a position hole to detachably hold the optical channel member.

In one or more embodiments, the elastic component is a spring arranged around the guiding rod and positioned between the first and second restriction portions.

In one or more embodiments, the optical tomography device further includes a plurality of distance sensors located on the carrier base, the least one sliding assembly is arranged radially relative to the through hole, each distance sensor is configured to detect a moving distance of a corresponding one of the least one sliding assembly.

In one or more embodiments, the optical tomography device further includes a plurality of blocking boards, each blocking board is located at an end of the guiding rod that is remote from the through hole, each distance sensor is configured to detect a moving distance of a corresponding one of the blocking boards.

In one or more embodiments, the at least one optical channel member has a first end and a second end, the first end is configured to contact an object to be observed, the optical tomography device further includes a plurality of optical sensors each optically coupled with the second end of a corresponding one of the at least one optical channel member and a processor configured to receive a plurality of optical signal data via the optical sensors; generate a profile of the object to be observed according to the moving distance; and generate a reconstructed optical image according to the profile and the optical signal data.

In sum, when the optical channel members are in contact with the object to be observed and continues to move toward the object to be observed, the elastic component of the sliding assembly causes the optical channel member, the guiding rod and the sliding block, to have a tolerance of deformation that makes the other optical channel members move toward and reach the object to be observed, thereby preventing the object, e.g., a patient's body part, from being over-squeezed and uncomfortable. Since there is no need to worry about the discomfort of the human body, the optical channel member can be in contact with a skin of the human body, and there is no gap between the optical channel member and the skin surface, which can reduce an interference of the measured signal, thereby improving the signal quality. The elastic component of the sliding assembly is configured to maintain relative positions of various portions of the sliding assembly such that the optical channel member and the guiding rod can be restored to their initial positions after the tomogram is performed. By sensing the moving distance of each sliding assembly by the distance sensor, the processor can determine a true contour or profile of the body part to be observed according to the moving distances. The optical sensor senses the object to be observed via the optical channel member, and the processor obtains a plurality of optical signal data via the optical sensor, and obtains a plurality of optical signal data to generate a reconstructed optical image according to the contour of the object to be observed. Therefore, the imaging accuracy of the reconstructed optical image can be improved.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 illustrates a perspective view of an optical tomography device in accordance with one embodiment of the present disclosure;

FIG. 2 illustrates an enlarged view of an optical channel member and a sliding assembly of the optical tomography device in FIG. 1;

FIG. 3 illustrates a cross-sectional view of the optical channel member and the sliding assembly taken along the line 3-3 in FIG. 2; and

FIG. 4 illustrates a functional block diagram of an optical tomography device in accordance with one embodiment of the present disclosure.

### DETAILED DESCRIPTION

It is to be noted that the following descriptions of preferred embodiments of this disclosure are presented herein for purpose of illustration and description only. It is not intended to be exhaustive or to be limited to the precise form disclosed. Also, it is also important to point out that there may be other features, elements, steps and parameters for implementing the embodiments of the present disclosure which are not specifically illustrated. Thus, the specification and the drawings are to be regard as an illustrative sense rather than a restrictive sense. Various modifications and similar arrangements may be provided by the persons skilled in the art within the spirit and scope of the present disclosure. In addition, the illustrations may not be necessarily be drawn to scale, and the identical elements of the embodiments are designated with the same reference numerals.

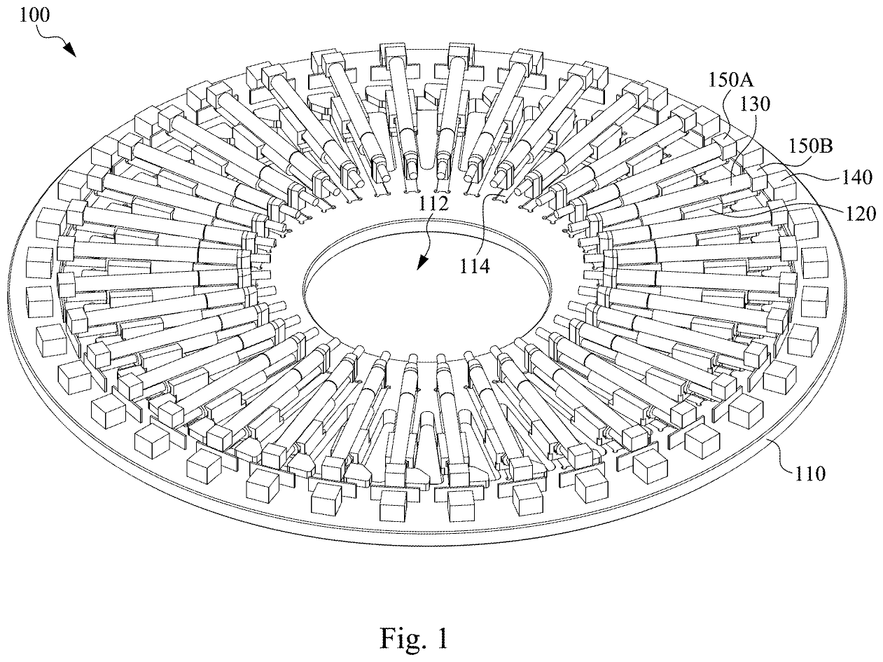

Reference is made to FIG. 1, which illustrates a perspective view of an optical tomography device 100 in accordance with one embodiment of the present disclosure. The optical tomography device 100 includes a carrier base 110, a plurality of sliding assemblies 120, a plurality of optical channel members 130, a plurality of distance sensors 140, a plurality of optical sensors 150A and a plurality of optical emitters 150B. The carrier base 110 has a central through hole 112 and a plurality of slide rails 114 that extend toward the through hole 112 and are arranged radially relative to the through hole 112. The through hole 112 is configured to hold an object to be observed, e.g., a body part such as a breast of a human body. Each sliding assembly 120 is slidably connected to a corresponding one of the slide rails 114 and thus slidable along the corresponding slide rail 114 on the carrier base 110. Each optical channel member 130 is coupled to a corresponding one of the sliding assemblies 120, and thus movable along with the corresponding sliding assembly 120 on the carrier base 110 to be closer to or away from the object to be observed. Of the optical channel members 130, some optical channel members 130 are equipped with optical emitters 150B to emit optical signals toward the object to be observed while other optical channel members 130 are equipped with optical sensors 150A to receive optical signals going through the object to be observed. In particular, the optical channel members 130 may be optical fiber channels, and the optical emitters 150B are configured to emit Near Infrared optical signals. A plurality of distance sensors 140 are located on a circumference edge of the carrier base 110 that are remote from the through hole 112. Each distance sensor 140 is configured to detect a moving distance of a corresponding sliding assembly 120.

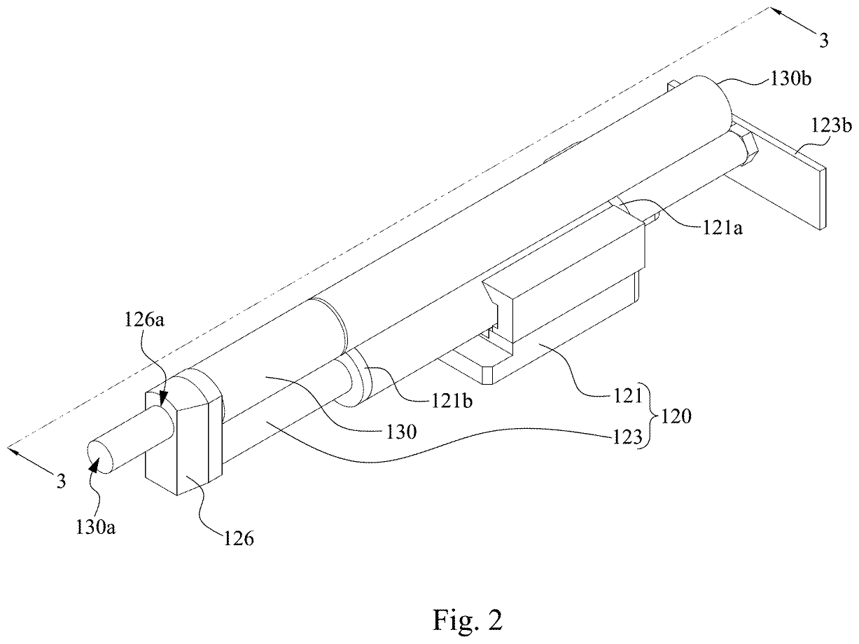

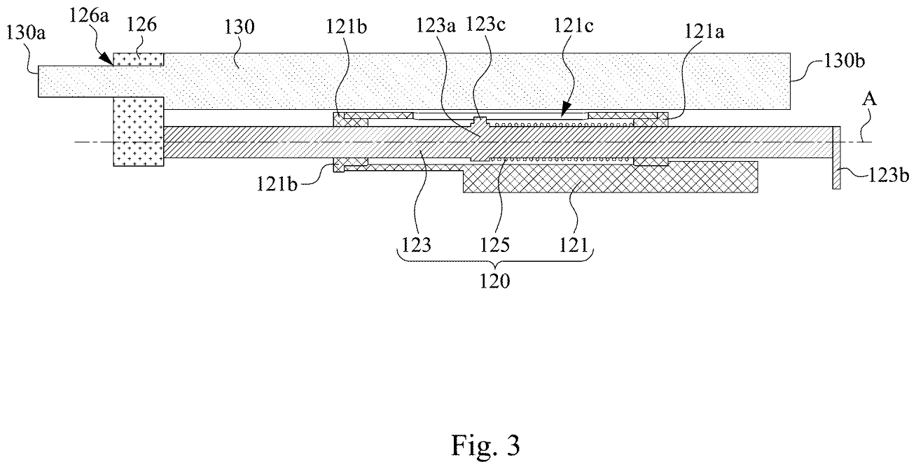

Reference is made to FIGS. 2-3. In one or more embodiments, optical channel member 130 has a first end 130a and a second end 130b. The first end 130a of the optical channel member 130 is configured to contact the object to be observed. The second end 130b of the optical channel member 130 is optically coupled with the optical sensor 150A or the optical emitter 150B.

In one or more embodiments, each sliding assembly 120 includes a sliding block 121, a guiding rod 123 and an elastic component 125. Each sliding block 121 is slidably connected to a corresponding slide rail 114 and has a first restriction portion 121a. Each guiding rod 123 is slidably connected to a corresponding sliding block 121 and has a second restriction portion 123a. The guiding rod 123 is configured to penetrate the first restriction portion 121a. The elastic component 125 is arranged around the guiding rod 123 and sandwiched between the first restriction portion 121a and the second restriction portion 123a, and compressed by the first restriction portion 121a and the second restriction portion 123a to be deformed. In particular, the elastic component 125 can be a spring.

Referring FIGS. 1-3 again, when the optical tomography device 100 is operated to perform a tomogram, each sliding assembly 120 is moved from an initial position along an axis A toward the through hole 112 so as to carry a corresponding optical channel member 130. When the optical channel member 130 is moved to be in contact with the object to be observed, the optical channel member 130, the guiding rod 123 and its second restriction portion 123a is no longer moved toward the through hole 112, but the sliding block 121 and its first restriction portion 121a is still moved toward the through hole 112 such that the elastic component 125 will be compressed by the first restriction portion 121a and the second restriction portion 123a. Because the optical channel member 130 is not slid along with the sliding block 121 toward the through hole 112, the object to be observed, e.g., a body part, is no longer over-squeezed by the optical channel member 130 such that no discomfort is caused for the patient.

In one or more embodiments, the elastic component 125 has two opposite ends connected to the first restriction portion 121a and the second restriction portion 123a such that the elastic component 125 provides a recovery force for the guiding rod 123 and the optical channel member 130. When the sliding block 121 is away from the through hole 112 and slid to the initial position on the carrier base 110, the elastic component 125 is configured to maintain a relative positions for the first restriction portion 121a and the second restriction portion 123a, the guiding rod 123 and optical channel member 130 are maintained at fixed positions relative to the sliding block 121 such that the sliding assembly 120 and the optical channel member 130 can be slid to the initial position on the carrier base 110.

Referring FIGS. 1-3 again, the sliding block 121 further includes a third restriction portion 121b. The guiding rod 123 is configured to penetrate the first and third restriction portions (121a, 121b), and slidably connected with the first and third restriction portions (121a, 121b). And the second restriction portion 123a is located between the first restriction portion 121a and the third restriction portion 121b. Because the guiding rod 123 is both supported by the first restriction portion 121a and the third restriction portion 121b, the guiding rod 123 can be reliably slid relative to the sliding block 121 along the axis A.

Referring FIGS. 1-3 again, the sliding block 121 has its first restriction portion 121a and third restriction portion 121b to collectively define an inner cavity to accommodate the guiding rod 123 and the elastic component 125. The elastic component 125 is located between the first restriction portion 121a and the second restriction portion 123a. The sliding block 121 has a guiding slot 121c communicated with the inner cavity and a protruding member 123c that is slidably connected within the guiding slot 121c. The guiding slot 121c is configured to extend toward the through hole 112, i.e., has a lengthwise direction extending toward the through hole 11 to prevent the guiding rod 123 from rotating about the axis A when the sliding assembly 120 moves toward or away from the through hole 112.

Referring FIGS. 2-3 again, the optical channel member 130 is coupled to a fastener 126 of the sliding assembly 120. The fastener 126 is located at an end of the guiding rod 123 that is immediately-adjacent to the through hole 112. The fastener 126 has a position hole 126a to detachably hold the optical channel member 130.

In other embodiments, a plurality of distance sensors 140 are configured to detect moving distances of the sliding assemblies 120 respectively. The distance sensors 140 are respectively located adjacent to the ends of the sliding assemblies that are remote from the through hole 112. In particular, each distance sensor 140 is located at an edge of the carrier base 110 in order to detect a moving distance of a corresponding guiding rod 123.

When the optical tomography device 100 is operated to perform a tomogram, each sliding assembly 120 is moved from an initial position along the axis A toward the through hole 112 so as to carry a corresponding optical channel member 130. When the optical channel member 130 is moved to be in contact with the object to be observed, the optical channel member 130, the guiding rod 123 and its second restriction portion 123a is no longer moved toward the through hole 112, an outer profile of the object determines a stop position of each guiding rod 123. Each distance sensor 140 is then configured to accurately detect a moving distance of a corresponding guiding rod 123 so as to accurately determine the outer profile of the object to be observed.

In other embodiments, a blocking board 123b may be installed at an end of the guiding rod 123 that is remote from the through hole 112. The blocking board 123b has a greater area than the end of the guiding rod 123 such that the distance sensor 140 can accurately detect a moving distance of the guiding rod 123 by detecting the blocking board 123b.

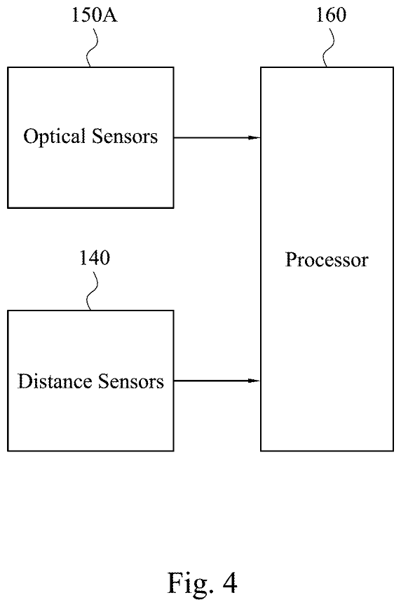

Referring FIGS. 1 and 4 again, in another embodiment, the optical tomography device 100 may include a plurality of distance sensors 140, a plurality of optical sensors 150A and a processor 160 that are coupled to the distance sensors 140 and the optical sensors 150A. The processor 160 is configured to receive or obtain a plurality of optical signal data via the optical sensors 150A, generate a profile of the object to be observed according to the moving distances of the sliding assemblies 120 detected by the distance sensors 140, and generate a reconstructed optical image according to the profile of the object to be observed and the optical signal data from the optical sensors 150A. In particular, the processor 160 is configured to combine the profile of the object generated by the moving distances of the sliding assemblies 120 detected by the distance sensors 140 and the optical signal data from the optical sensors 150A to accurately generate a reconstructed optical image for the object to be observed.

According to aforementioned embodiments, when the optical channel members are in contact with the object to be observed and continues to move toward the object to be observed, the elastic component of the sliding assembly causes the optical channel member, the guiding rod and the sliding block, to have a tolerance of deformation that makes the other optical channel members move toward and reach the object to be observed, thereby preventing the object, e.g., a patient's body part, from being over-squeezed and uncomfortable. Since there is no need to worry about the discomfort of the human body, the optical channel member can be in contact with a skin of the human body, and there is no gap between the optical channel member and the skin surface, which can reduce an interference of the measured signal, thereby improving the signal quality. The elastic component of the sliding assembly is configured to maintain relative positions of various portions of the sliding assembly such that the optical channel member and the guiding rod can be restored to their initial positions after the tomogram is performed. By sensing the moving distance of each sliding assembly by the distance sensor, the processor can determine a true contour or profile of the body part to be observed according to the moving distances. The optical sensor senses the object to be observed via the optical channel member, and the processor obtains a plurality of optical signal data via the optical sensor, and obtains a plurality of optical signal data to generate a reconstructed optical image according to the contour of the object to be observed. Therefore, the imaging accuracy of the reconstructed optical image can be improved.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. An optical tomography device comprising:
a carrier base having a through hole;
at least one slide rail disposed on the carrier base and extended toward the through hole;
at least one sliding assembly comprising:
a sliding block slidably connected to the at least one slide rail and having a first restriction portion;
a guiding rod slidably connected to the sliding block and having a second restriction portion; and
an elastic component configured to be deformed while being pressed by the first and second restriction portions; and
at least one optical channel member coupled with the guiding rod.

2. The optical tomography device of claim 1, wherein the sliding block has a third restriction portion, the guiding rod is configured to penetrate the first and third restriction portions, and slidably connected with the first and third restriction portions.

3. The optical tomography device of claim 1, wherein the sliding block has an inner cavity to accommodate the elastic component.

4. The optical tomography device of claim 3, wherein the sliding block has a guiding slot communicated with the inner cavity and a protruding member slidably connected within the guiding slot.

5. The optical tomography device of claim 4, wherein the guiding slot is configured to extend toward the through hole.

6. The optical tomography device of claim 1, wherein the at least one sliding assembly further comprise a fastener disposed at an end of the guiding rod that is immediately-adjacent to the through hole, the fastener has a position hole to detachably hold the optical channel member.

7. The optical tomography device of claim 1, wherein the elastic component is a spring disposed around the guiding rod and between the first and second restriction portions.

8. The optical tomography device of claim 1 further comprising a plurality of distance sensors disposed on the carrier base, the at least one sliding assembly is disposed radially relative to the through hole, each distance sensor is configured to detect a moving distance of a corresponding one of the at least one sliding assembly.

9. The optical tomography device of claim 8 further comprising a plurality of blocking boards, each blocking board is disposed at an end of the guiding rod that is remote from the through hole, each distance sensor is configured to detect a moving distance of a corresponding one of the blocking boards.

10. The optical tomography device of claim 9, wherein the at least one optical channel member has a first end and a second end, the first end is configured to contact an object to be observed, the optical tomography device further comprises:
a plurality of optical sensors each optically coupled with the second end of a corresponding one of the at least one optical channel member; and
a processor configured to:
receive a plurality of optical signal data via the optical sensors;
generate a profile of the object to be observed according to the moving distance; and
generate a reconstructed optical image according to the profile and the optical signal data.
